(19) United States
(12) Patent Application Publication
(10) Pub. No.: US 2012/0157741 A1
(43) Pub. Date: Jun. 21, 2012

(54) Synthesis of Silicoaluminophosphate Having Lev Framework-Type

(76) Inventors: Guang Cao, Princeton, NJ (US); Matu J. Shah, Hackettstown, NJ (US)

(21) Appl. No.: 12/972,202

(22) Filed: Dec. 17, 2010

(51) Int. Cl. C07C 1/20; C01B 39/54

(52) U.S. Cl. 585/640; 423/704; 423/700

(57) ABSTRACT

A process for producing a silicoaluminophosphate molecular sieve having the LEV framework-type employs at least one source of triethylmethylammonium, R⁺, ions; as a templating agent. The resultant silicoaluminophosphate molecular sieve is useful as a catalyst in the conversion of methanol to olefins.

### FIELD

This invention relates to the synthesis of a silicoaluminophosphate having the LEV framework-type and its use as a catalyst in the conversion of methanol to olefins.

### BACKGROUND

Silicoaluminophosphate (SAPO) molecular sieves contain a three-dimensional microporous crystalline framework structure of [SiO₄], [AlO₄] and [PO₄] corner sharing tetrahedral units. SAPOs, and particularly SAPOs having a small pore size of 5 Angstrom or less, are some of the most useful catalysts currently known for converting methanol to olefin(s). Among the silicoaluminophosphate molecular sieves that have been demonstrated to have activity in methanol to olefin (MTO) conversion are SAPOs with the framework types of ERI (SAPO-17), CHA (SAPO-34, SAPO-44, and SAPO-47), and LEV (SAPO-35). Although SAPO-34 is generally the most preferred MTO catalyst, because of its selectivity to ethylene and propylene, SAPO-35 is also an active MTO catalyst and remains of interest where different light olefin selectivity is desirable. For example, the following table taken from an article by Stephen Wilson and Paul Barger in Microporous and Mesoporous Materials, Vol. 29 (1999), pp. 117-126 compares the product slate obtained in the conversion of methanol to hydrocarbons at 648° K over a variety of silicoaluminophosphate molecular sieves.

| SAPO | 17 | 34 | 44 | 16 | 35 |
|---|---|---|---|---|---|
| C₂⁼ | 36.5 | 35 | 17.7 | 0.5 | 42.8 |
| C₂ | 0.5 | 0.6 | 6.3 | Trace | 0.4 |
| C₃⁼ | 29.3 | 43.0 | 13.3 | 0.6 | 31.2 |
| C₃ | Trace | 0.4 | 9.5 | Trace | 1.3 |
| C₄⁼ | 12.2 | 15.8 | 7.4 | Trace | 8.0 |
| C₅s | 4.9 | 3.6 | 1.1 | ND | 2.9 |
| C₆s | 2.0 | Trace | ND | ND | 1.4 |
| C₁ | 2.9 | 1.5 | 5.5 | Nd | 11.5 |
| CO₂ | 0.2 | 0.2 | 2.8 | ND | 0.6 |
| DME | 0.0 | 0.0 | 36.4 | 98.9 | 0.0 |
| TOS (hrs) | 4.7 | 6.3 | 1.0 | 2.0 | 1.0 |
| MeOH WHSV (hr⁻¹) | 0.86 | 1.17 | 0.85 | 0.87 | 2.60 |
| H₂O WHSV (hr⁻¹) | 2.00 | 2.73 | 1.99 | 2.03 | 2.43 |
| Conversion (%) | 100 | 100 | 45 | 53 | 100 |

SAPO-35 is isostructural with the zeolite levynite (LEV) and its synthesis, using quinuclidine as a templating agent, was first reported in U.S. Pat. No. 4,440,871. In addition, Lohse et al. have reported that SAPO-35 can be prepared with cyclohexylamine as a templating agent, see Crystal Research and Technology, Vol. 28 (1993), Issue 8, pp. 1101-1107. Further, Venkatathri et al. disclose that SAPO-35 can be synthesized in a non-aqueous gel using hexamethyleneimine as a templating agent, see J. Chem. Soc., Faraday Trans, 1997, Vol. 93, Issue 18, pp. 3411-3415.

However, although quinuclidine is very specific in its ability to induce the crystallization of SAPO-35, it is prohibitively expensive for use in commercial production. On the other hand, although cyclohexylamine and hexamethyleneimine are less expensive than quinuclidine, they are not structure specific templates. For example, cyclohexylamine can direct the synthesis of a number of different SAPO structures, such as SAPO-17 and SAPO-44, in addition to SAPO-35. As a result, producing pure phase materials with such non-structure specific templates requires rigorous control over the synthesis conditions.

There is therefore interest in finding alternative templating agents for the synthesis of SAPO-35.

According to the present invention, it has now been found that triethylmethylammonium cations are generally effective and inexpensive templating agents for the synthesis of SAPO-35. Moreover, by conducting the synthesis in the presence of levynite zeolite seeds, it is possible to produce SAPO-35 with controlled crystal size and improved MTO performance.

### SUMMARY

In one aspect, the invention resides in a process for producing a silicoaluminophosphate molecular sieve having the LEV framework-type, the process comprising:

(a) providing a reaction mixture comprising at least one source of aluminum, at least one source of phosphorus, at least one source of silicon and at least one source of triethylmethylammonium, R⁺, ions; and

(b) crystallizing said reaction mixture under conditions effective to produce said silicoaluminophosphate having the LEV framework-type.

Conveniently, the molar ratio of R⁺ ions to aluminum in the reaction mixture, expressed as the molar ratio of R⁺ ions to alumina (Al₂O₃), is within the range of from about 1:1 to about 2:1.

Conveniently, the reaction mixture also contains seeds, typically from about 0.01 ppm by weight to about 10,000 ppm by weight, such as from about 100 ppm by weight to about 5,000 by weight, of seeds.

In one embodiment, the seeds comprise a crystalline aluminosilicate material (zeolite) having a LEV framework-type.

Conveniently, the synthesis mixture comprises a source of silicon present in an amount such that said mixture has a non-zero Si:Al₂ molar ratio up to about 0.5.

Conveniently, said conditions in (b) include a temperature of about 130° C. to about 220° C. for a time of about 20 to about 200 hours.

In a further aspect, the invention resides in a silicoaluminophosphate molecular sieve having the LEV framework-type comprising triethylmethylammonium, R⁺, ions within its intra-crystalline structure.

In yet a further aspect, the invention resides in a process for producing olefins comprising contacting an organic oxygenate compound under oxygenate conversion conditions with the catalyst composition comprising a calcined form of the silicoaluminophosphate molecular sieve described herein.

### BRIEF DESCRIPTION OF THE DRAWINGS

FIG. 1 provides X-ray diffraction patterns of the as-synthesized silicoaluminophosphate molecular sieves produced in the process of Example 1 using varying amounts of triethylmethylammonium hydroxide as a templating agent.

FIG. 2 provides X-ray diffraction patterns of the as-synthesized silicoaluminophosphate molecular sieves produced in the process of Example 2 using triethylmethylammonium hydroxide as a templating agent and varying Si:Al₂ molar ratios with and without seeds.

FIG. 3 provides X-ray diffraction patterns of the as-synthesized silicoaluminophosphate molecular sieves produced in the process of Example 3 using quinuclidine as a templating agent and varying Si:Al₂ molar ratios with and without seeds.

FIG. 4 provides scanning electron micrographs of the products of Examples 2 and 3.

### DETAILED DESCRIPTION OF THE EMBODIMENTS

Described herein is a process for producing a silicoaluminophosphate (SAPO) molecular sieve having the LEV framework in which at least one source of triethylmethylammonium, R⁺, ions is employed as a templating agent.

SAPOs with the LEV framework-type structure have a two-dimensional arrangement of pores defined by eight-membered rings of interconnected oxygen atoms with average cross-sectional dimensions of 3.6 Å by 4.8 Å. Such materials may be characterized by their unique X-ray diffraction pattern which has at least the reflections in the 5 to 25 (2θ) range as shown in Table 1 below:

TABLE 1

| 2θ (CuKα) | I % |
|---|---|
| 8.66(±0.05) | 20 |
| 10.97(±0.05) | 65 |
| 13.38(±0.04) | 35 |
| 15.94(±0.04) | 10 |
| 17.31(±0.04) | 70 |
| 17.77(±0.04) | 10 |
| 20.97(±0.03) | 45 |
| 21.92(±0.03) | 100 |
| 23.24(±0.03) | 20 |
| 24.93(±0.03) | 10 |
| 26.87(±0.02) | 15 |
| 28.45(±0.02) | 30 |
| 29.09(±0.02) | 10 |
| 31.55(±0.01) | 5 |
| 32.13(±0.01) | 35 |
| 34.39(±0.01) | 10 |
| 35.80(±0.01) | 5 |

The X-ray diffraction data referred to in Table 1 are collected with a SCINTAG X2 X-Ray Powder Diffractometer (Scintag Inc., USA), using copper K-alpha radiation. The diffraction data are recorded by step-scanning at 0.02 degrees of two-theta, where theta is the Bragg angle, and a counting time of 1 second for each step. Prior to recording of each experimental X-ray diffraction pattern, the sample must be in the anhydrous state and free of any template used in its synthesis, since the simulated patterns are calculated using only framework-type atoms, not extra-framework material such as water or template in the cavities. Given the sensitivity of silicoaluminophosphate materials to water at recording temperatures, the molecular sieve samples are calcined after preparation and kept moisture-free according to the following procedure.

About 2 grams of each molecular sieve sample are heated in an oven from room temperature under a flow of nitrogen at a rate of 3° C./minute to 200° C. and, while retaining the nitrogen flow, the sample is held at 200° C. for 30 minutes and the temperature of the oven is then raised at a rate of 2° C./minute to 650° C. The sample is then retained at 650° C. for 8 hours, the first 5 hours being under nitrogen and the final 3 hours being under air. The oven is then cooled to 200° C. at 30° C./minute and, when the XRD pattern is to be recorded, the sample is transferred from the oven directly to a sample holder and covered with Mylar foil to prevent rehydration.

The LEV framework-type type silicoaluminophosphate molecular sieve described herein is synthesized by the hydrothermal crystallization of a source of alumina, a source of phosphorus, a source of silica and a source of triethylmethylammonium, R⁺, ions as an organic templating agent. In particular, an aqueous reaction mixture comprising sources of silica, alumina and phosphorus, together with triethylmethylammonium, R⁺, ions and optionally seeds from another or the same framework-type molecular sieve, is placed in a sealed pressure vessel, optionally lined with an inert plastic such as polytetrafluoroethylene, and heated at a crystallization temperature until the desired crystalline material is formed. Typically, the reaction mixture has a composition, in terms of mole ratios of oxides, within the ranges indicated in Table 2 below.

TABLE 2

| Reactants | Useful | Typical |
|---|---|---|
| P₂O₅/Al₂O₃ | 0.8-1.5 | 0.9-1.3 |
| SiO₂/Al₂O₃ | 0-0.6 | 0.05-0.35 |
| H₂O/Al₂O₃ | 30-80 | 35-60 |
| R⁺/Al₂O₃ | 0.8-3.0 | 1.0-2.0 |
| R⁺OH⁻/P₂O₃ | 0.8-2.0 | 0.9-2.0 |

Non-limiting examples of suitable silica sources include silicates, fumed silica, for example, Aerosil-200 available from Degussa Inc., New York, N.Y., and CAB-O-SIL M-5, organosilicon compounds such as tetraalkyl orthosilicates, for example, tetramethyl orthosilicate (TMOS) and tetraethylorthosilicate (TEOS), colloidal silicas or aqueous suspensions thereof, for example Ludox HS-40 sol available from E.I. du Pont de Nemours, Wilmington, Del., silicic acid or any combination thereof.

Non-limiting examples of suitable alumina sources include organoaluminum compounds such as aluminum alkoxides, for example aluminum isopropoxide, and inorganic aluminum sources, such as aluminum phosphate, aluminum hydroxide, sodium aluminate, pseudo-boehmite, gibbsite and aluminum trichloride, or any combination thereof. Preferred sources are inorganic aluminum compounds, such as hydrated aluminum oxides and particularly boehmite and pseudoboehmite.

Non-limiting examples of suitable phosphorus sources, which may also include aluminum-containing phosphorus compositions, include phosphoric acid, organic phosphates such as triethyl phosphate, and crystalline or amorphous aluminophosphates such as AlPO₄, phosphorus salts, or combinations thereof. A preferred source of phosphorus is phosphoric acid.

Non-limiting examples of suitable sources of triethylmethylammonium, R⁺, ions include triethylmethylammonium hydroxide and triethylmethylammonium salts, such as halide salts.

Synthesis of the desired LEV framework-type silicoaluminophosphate may be facilitated by the presence of at least 0.01 ppm by weight, such as at least 10 ppm by weight, for example at least 100 ppm by weight, up to 10,000 ppm by weight, conveniently up to about 5,000 by weight, of seeds. The seed crystals can be homostructural with the desired crystalline material and can have the same composition as the desired crystalline material, for example the product of a previous synthesis. Preferably, however, the seed crystals have a different composition from the desired crystalline material of the present invention and in particular comprise a crystalline aluminosilicate material (zeolite) having a LEV framework-type. The production of colloidal seed suspensions and their use in the synthesis of molecular sieves are disclosed in, for example, International Publication Nos. WO 00/06493 and WO 00/06494.

After combining all the components of the reaction mixture, the mixture is heated, preferably under autogenous pressure, to a temperature in the range of from 130° C. to about 220° C., for example from about 150° C. to about 200° C. The time required to form the crystalline product is usually dependent on the temperature and typically varies from about 20 hours to around 200 hours, such as from about 48 hours to around 168 hours. The hydrothermal crystallization may be carried out without or, more preferably, with agitation.

Once the crystalline molecular sieve product is formed, usually in a slurry state, it may be recovered by any standard techniques well known in the art, for example, by centrifugation or filtration. The recovered crystalline product may then be washed, such as with water, and then dried, such as in air.

As a result of the synthesis process, the crystalline product recovered from the reaction mixture contains within its pores at least a portion of the organic templating agent used in the synthesis. In a preferred embodiment, activation is performed in such a manner that the organic templating agent is removed from the molecular sieve, leaving active catalytic sites within the microporous channels of the molecular sieve open for contact with a feedstock. The activation process is typically accomplished by calcining, or essentially heating the molecular sieve comprising the template at a temperature of from about 200° C. to about 800° C. in the presence of an oxygen-containing gas. In some cases, it may be desirable to heat the molecular sieve in an environment having a low or zero oxygen concentration. This type of process can be used to effect partial or complete removal of the organic templating agent from the intracrystalline pore system of the molecular sieve.

The silicoaluminophosphate molecular sieve produced by the present synthesis method is particularly intended for use as organic conversion catalysts. Before use in catalysis, the molecular sieve will normally be formulated into catalyst compositions by combination with other materials, such as binders and/or matrix materials, which provide additional hardness or catalytic activity to the finished catalyst.

Materials which can be blended with the molecular sieve can be various inert or catalytically active materials. These materials include compositions such as kaolin and other clays, various forms of rare earth metals, other non-zeolite catalyst components, zeolite catalyst components, alumina or alumina sol, titania, zirconia, quartz, silica or silica sol, and mixtures thereof. These components are also effective in reducing overall catalyst cost, acting as a thermal sink to assist in heat shielding the catalyst during regeneration, densifying the catalyst and increasing catalyst strength. When blended with such components, the amount of molecular sieve contained in the final catalyst product ranges from 10 to 90 weight percent of the total catalyst, preferably 20 to 80 weight percent of the total catalyst composition.

The silicoaluminophosphate molecular sieve described herein is useful as a catalyst in a variety of processes including cracking of, for example, a naphtha feed to light olefin(s) or higher molecular weight (MW) hydrocarbons to lower MW hydrocarbons; hydrocracking of, for example, heavy petroleum and/or cyclic feedstock; polymerization of, for example, one or more olefin(s) to produce a polymer product; reforming; hydrogenation; dehydrogenation; dewaxing of, for example, hydrocarbons to remove straight chain paraffins; absorption of light hydrocarbons such as methane, ethane, ethylene, propylene, acetylene, and CO₂.

The silicoaluminophosphate molecular sieve produced by the present method is particularly suitable for use as a catalyst in the conversion of oxygenates to olefins. As used herein, the term “oxygenates” is defined to include, but is not necessarily limited to aliphatic alcohols, ethers, carbonyl compounds (aldehydes, ketones, carboxylic acids, carbonates, and the like), and also compounds containing hetero-atoms, such as, halides, mercaptans, sulfides, amines, and mixtures thereof. The aliphatic moiety will normally contain from about 1 to about 10 carbon atoms, such as from about 1 to about 4 carbon atoms.

Representative oxygenates include lower straight chain or branched aliphatic alcohols, their unsaturated counterparts, and their nitrogen, halogen and sulfur analogues. Examples of suitable oxygenate compounds include methanol; ethanol; n-propanol; isopropanol; C₄-C₁₀ alcohols; methyl ethyl ether; dimethyl ether; diethyl ether; di-isopropyl ether; methyl mercaptan; methyl sulfide; methyl amine; ethyl mercaptan; di-ethyl sulfide; di-ethyl amine; ethyl chloride; formaldehyde; di-methyl carbonate; di-methyl ketone; acetic acid; n-alkyl amines, n-alkyl halides, n-alkyl sulfides having n-alkyl groups of comprising the range of from about 3 to about 10 carbon atoms; and mixtures thereof. Particularly suitable oxygenate compounds are methanol, dimethyl ether, or mixtures thereof, most preferably methanol. As used herein, the term “oxygenate” designates only the organic material used as the feed. The total charge of feed to the reaction zone may contain additional compounds, such as diluents.

In the present oxygenate conversion process, a feedstock comprising an organic oxygenate, optionally with one or more diluents, is contacted in the vapor phase in a reaction zone with a catalyst comprising the molecular sieve described herein at effective process conditions so as to produce the desired olefins. Alternatively, the process may be carried out in a liquid or a mixed vapor/liquid phase. When the process is carried out in the liquid phase or a mixed vapor/liquid phase, different conversion rates and selectivities of feedstock-to-product may result depending upon the catalyst and the reaction conditions.

When present, the diluent(s) is generally non-reactive to the feedstock or molecular sieve catalyst composition and is typically used to reduce the concentration of the oxygenate in the feedstock. Non-limiting examples of suitable diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred. Diluent(s) may comprise from about 1 mol % to about 99 mol % of the total feed mixture.

The temperature employed in the oxygenate conversion process may vary over a wide range, such as from about 200° C. to about 1000° C., for example from about 250° C. to about 800° C., including from about 250° C. to about 750° C., conveniently from about 300° C. to about 650° C., typically from about 350° C. to about 600° C. and particularly from about 400° C. to about 600° C.

Light olefin products will form, although not necessarily in optimum amounts, at a wide range of pressures, including but not limited to autogenous pressures and pressures in the range of from about 0.1 kPa to about 10 MPa. Conveniently, the pressure is in the range of from about 7 kPa to about 5 MPa, such as in the range of from about 50 kPa to about 1 MPa. The foregoing pressures are exclusive of diluent, if any is present, and refer to the partial pressure of the feedstock as it relates to oxygenate compounds and/or mixtures thereof. Lower and upper extremes of pressure may adversely affect selectivity, conversion, coking rate, and/or reaction rate; however, light olefins such as ethylene still may form.

The process should be continued for a period of time sufficient to produce the desired olefin products. The reaction time may vary from tenths of seconds to a number of hours. The reaction time is largely determined by the reaction temperature, the pressure, the catalyst selected, the weight hourly space velocity, the phase (liquid or vapor) and the selected process design characteristics.

A wide range of weight hourly space velocities (WHSV) for the feedstock will function in the present process. WHSV is defined as weight of feed (excluding diluent) per hour per weight of a total reaction volume of molecular sieve catalyst (excluding inerts and/or fillers). The WHSV generally should be in the range of from about 0.01 hr⁻¹ to about 500 hr⁻¹, such as in the range of from about 0.5 hr⁻¹ to about 300 hr⁻¹, for example in the range of from about 0.1 hr⁻¹ to about 200 hr⁻¹.

A practical embodiment of a reactor system for the oxygenate conversion process is a circulating fluid bed reactor with continuous regeneration, similar to a modern fluid catalytic cracker. Fixed beds are generally not preferred for the process because oxygenate to olefin conversion is a highly exothermic process which requires several stages with intercoolers or other cooling devices. The reaction also results in a high pressure drop due to the production of low pressure, low density gas.

Because the catalyst must be regenerated frequently, the reactor should allow easy removal of a portion of the catalyst to a regenerator, where the catalyst is subjected to a regeneration medium, such as a gas comprising oxygen, for example air, to burn off coke from the catalyst, which restores the catalyst activity. The conditions of temperature, oxygen partial pressure, and residence time in the regenerator should be selected to achieve a coke content on regenerated catalyst of less than about 0.5 wt %. At least a portion of the regenerated catalyst should be returned to the reactor.

Using the various oxygenate feedstocks discussed above, particularly a feedstock containing methanol, a catalyst composition comprising the molecular sieve described herein is effective to convert the feedstock primarily into one or more olefin(s). The olefin(s) produced typically have from 2 to 30 carbon atoms, preferably 2 to 8 carbon atoms, more preferably 2 to 6 carbon atoms, still more preferably 2 to 4 carbons atoms, and most preferably are ethylene and/or propylene. The resultant olefins can be separated from the oxygenate conversion product for sale or can be fed to a downstream process for converting the olefins to, for example, polymers.

The invention will now be more particularly described with reference to the following non-limiting Examples and the accompanying drawing.

In the Examples, X-ray powder diffractograms were recorded on a Siemens D500 diffractometer with a voltage of 40 kV and current of 30 mA, using a Cu target and Ni-filter (λ=0.154 nm). Elemental analysis of Al, Si, and P was performed using Inductively Coupled Plasma (ICP) spectroscopy.

### Example 1

### Synthesis of SAPO-35 and SAPO-18 Using TEMAOH Template

The following ingredients were mixed in sequence and blended into a uniform gel using a microhomogenizer (Tissue Tearor Model 98730 available from Biospec Products, Inc, USA): 6.73 g of 85 wt % H₃PO₄ (Aldrich Chemical Company), 7.19 g deionized H₂O, 4.03 g Catapal™ A (71.5 wt % Al₂O₃, available from CONDEA Vista Company, Texas, USA), 0.39 g Cabosil™ silica (Cabot Corporation, Illinois, USA), and 11.67 g 40% triethylmethylammonium hydroxide (TEMAOH) (Sachem Company, USA). The molar ratio of the ingredients was as follows:

1.2TEMAOH:1.0Al₂O₃:0.2SiO₂:1.15P₂O₅:34H₂O

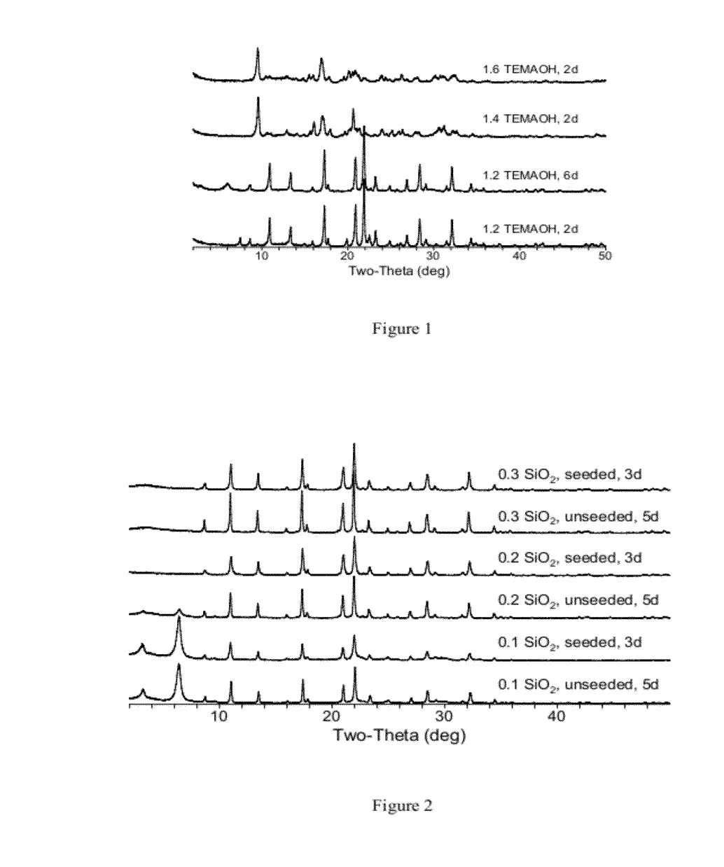

The gel (pH=4-5) was divided into two equal portions and placed into 23-mL Teflon-lined stainless steel autoclaves. The autoclaves were heated to 170° C., one for 2 days and the other for 6 days, in an oven while being tumbled at 20 rpm. The solid products were centrifuged (supernatant pH=7) and washed several times with deionized water, then dried in a 60° C. vacuum oven overnight. X-ray powder patterns of the as-synthesized materials (FIG. 1) indicated that both products were SAPO-35 with some impurity.

The above experiment was repeated, except that 1.4 and 1.6 mole of TEMAOH template (per mole of Al₂O₃) were used. The products so synthesized were mostly SAPO-18, as also shown in FIG. 1.

### Example 2

### Synthesis of Pure SAPO-35 with TEMAOH

The procedure of Example 1 was repeated to prepare three batches (30 g each) of gel having the following molar compositions:

1.5TEMAOH:1.0Al₂O₃:xSiO₂:1.0P₂O₅:45H₂O (x=0.1, 0.2, 0.3)

Each batch of gel was divided into two equal portions. To one of them was added 100 ppm of colloidal LEV aluminosilicate seeds and none to the other. The seeded gel mixtures were heated at 170° C. for 3 days in the same way as in Example 1, while the unseeded gel mixtures were heated at 170° C. for 5 days. The XRD patterns of the as-synthesized product indicate that pure SAPO-35 was made from the synthesis mixtures for which x=0.3, whether seeded or unseeded, and from the seeded mixture for which x=0.2. The other three synthesis mixtures produced SAPO-35 with some impurities. See FIG. 2. These results show that Si/Al₂O₃ ratio>0.1 and seeding favor the formation of pure SAPO-35. Seeding also caused noticeable broadening in XRD peaks, indicating reduction of crystal size.

### Example 3 (Comparative)

### SAPO-35 Synthesized with Quinuclidine

The procedure of Example 2 was repeated but using quinuclidine as the templating agent to produce gels with the following molar composition:

1.5quinuclidine:1.0Al₂O₃:xSiO₂:1.0P₂O₅:40H₂O (x=0.1, 0.2, 0.3)

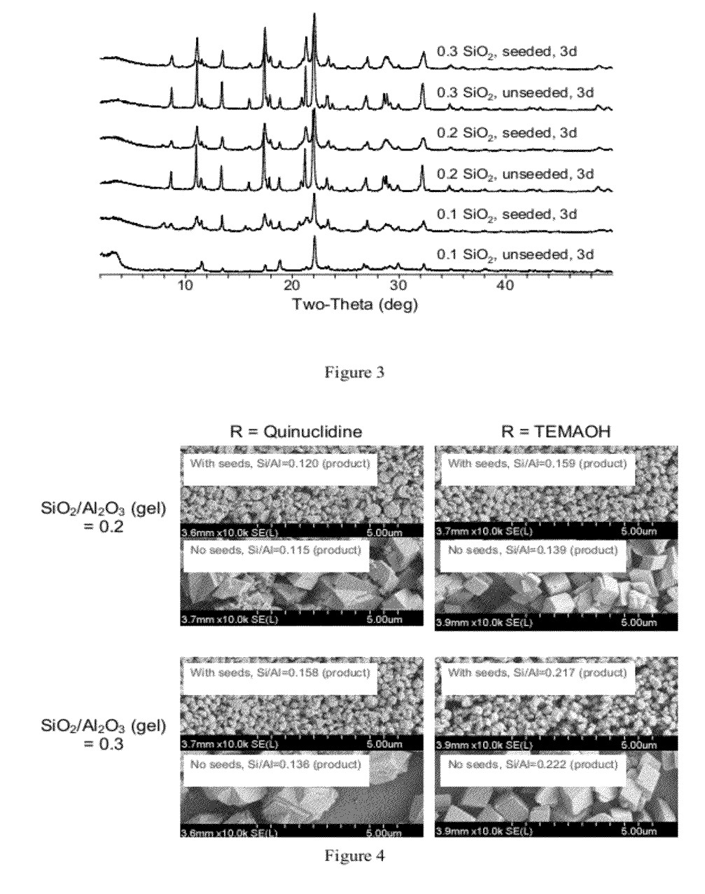

Each gel was heated at 170° C. for 3 days. The XRD patterns of the as-synthesized products indicate that pure SAPO-35 was made from four of the six synthesis mixtures, see FIG. 3. These results show that, similarly to TEMAOH as template, Si/Al₂O₃ ratios >0.1 and seeding favor the formation of SAPO-35 using quinuclidine as the templating agent. Seeding also caused noticeable broadening in XRD peaks, indicating reduction of crystal size. FIG. 4 shows the crystal size and morphology of the SAPO-35 samples described in Examples 2 and 3. The crystals are larger than 2 μm without seeds and smaller than 0.5 μm with seeds.

### Example 4

### MTO Testing Results

The Methanol-To-Olefins (MTO) reaction was carried out in a fixed-bed microreactor and, during the test, methanol was fed at a preset pressure and rate to a stainless steel reactor tube housed in an isothermally heated zone. The reactor tube contained about 20 mg weighed and sized granules of the catalyst sample (20-40 mesh by press-and-screen method). The catalyst had been calcined (ramp to 600° C. and hold for up to three hours in air) before being loaded to the reactor tube, and was activated for 30 minutes at 500° C. in flowing nitrogen before methanol was admitted. The product effluent was sampled, at different times during the run, with a twelve-port sampling loop while the catalyst was continuously deactivating. The effluent sample in each port was analyzed with a Gas Chromatograph equipped with an FID detector.

The testing conditions were as follows: the temperature was 475° C. and the pressure of methanol was 40 psia (276 kPa). The feed rate in weight hourly space velocity (WHSV) was 100/h. Cumulative conversion of methanol was expressed as grams of methanol converted per gram of sieve catalyst (CMCPS). On-stream lifetime refers to the CMCPS when methanol conversion has dropped to 10%. The product selectivity was reported as averages over the entire conversion range, rather than from a single point in effluent composition.

Table 3 shows a comparison of the MTO performance of the products of Examples 2 and 3. The data indicate that (1) smaller crystals obtained with seeding give better performance in having longer on-stream lifetime (total MeOH converted) and higher light olefin (C2= plus C3=) selectivity; and (2) SAPO-35 derived from TEMAOH shows generally better MTO performance than quinuclidine derived SAPO-35. The highest selectivity for ethylene plus propylene (55.1%) was obtained with TEMAOH-templated, seeded SAPO-35 having Si/Al molar ratio=0.200, whereas the quinuclidine-templated, seeded SAPO-35 having the closest Si/Al molar ratio (0.159) had selectivity for ethylene plus propylene of 49.9%.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

TABLE 3

| | Si/Al | Total g | | | | | | | | | | |
|---|---|---|---|---|---|---|---|---|---|---|---|---|
| | Molar | MeOH | | Initial | | | | | | | | |
| Sample Designation | Ratio | Converted | C₂⁼ + C₃⁼ | Conv | C2=/C3= | C₄⁺ | CH4 | C2= | C2o | C3= | C3o | C4's |
| Quinuclidine, 0.2 Si, LEV seeds | 0.120 | 0.6 | 17.8 | 4.5% | 1.26 | 3.8 | 18.6 | 9.9 | 0.0 | 7.9 | 0.4 | 2.3 |
| Quinuclidine, 0.3 Si, LEV seeds | 0.159 | 2.0 | 49.9 | 92.4% | 1.01 | 24.4 | 4.9 | 25.1 | 1.4 | 24.8 | 2.5 | 10.5 |
| Quinuclidine, 0.3 Si, No seeds | 0.136 | 1.4 | 37.8 | 88.8% | 0.75 | 23.3 | 7.1 | 16.3 | 1.4 | 21.6 | 2.1 | 13.6 |
| TEMAOH, 0.2 Si, LEV seeds | 0.200 | 2.2 | 55.1 | 99.3% | 1.03 | 16.8 | 5.4 | 27.9 | 3.5 | 27.2 | 4.5 | 7.7 |
| TEMAOH, 0.3 Si, LEV seeds | 0.217 | 1.7 | 50.7 | 99.4% | 0.96 | 14.6 | 6.6 | 24.8 | 5.2 | 25.9 | 4.7 | 6.6 |
| TEMAOH, 0.3 Si, No seeds | 0.222 | 1.2 | 48.8 | 98.1% | 0.66 | 15.8 | 8.1 | 19.5 | 2.7 | 29.4 | 1.7 | 8.1 |

What is claimed is:

1. A process for producing a silicoaluminophosphate molecular sieve having the LEV framework-type, the process comprising:
(a) providing a reaction mixture comprising at least one source of aluminum, at least one source of phosphorus, at least one source of silicon and at least one source of triethylmethylammonium, R⁺, ions; and
(b) crystallizing said reaction mixture under conditions effective to produce said silicoaluminophosphate molecular sieve.

2. The process of claim 1, wherein the molar ratio of R⁺ ions to aluminum in the reaction mixture, expressed as the molar ratio of R⁺ ions to alumina (Al₂O₃), is within the range of from about 1:1 to about 2:1.

3. The process of claim 1, wherein the reaction mixture also contains seeds.

4. The process of claim 1, wherein said reaction mixture comprises from about 0.01 ppm by weight to about 10,000 ppm by weight of seeds.

5. The process of claim 1, wherein said reaction mixture comprises from about 100 ppm by weight to about 5,000 by weight of seeds.

6. The process of claim 3, wherein said seeds comprise a crystalline aluminosilicate material (zeolite) having a LEV framework-type.

7. The process of claim 1, wherein said synthesis mixture comprises a source of silicon present in an amount such that said mixture has a non-zero Si:Al₂ molar ratio up to about 0.5.

8. The process of claim 1 wherein said conditions in (b) include a temperature of about 130° C. to about 220° C. for a time of about 20 to about 200 hours.

9. A silicoaluminophosphate molecular sieve having the LEV framework-type comprising triethylmethylammonium, R⁺, ions within its intra-crystalline structure.

10. A catalyst composition comprising a calcined form of the molecular sieve of claim 9.

11. A catalyst composition comprising SAPO-35 as produced by the process of claim 1.

12. A process for producing olefins comprising contacting an organic oxygenate compound under oxygenate conversion conditions with the catalyst composition of claim 10.

13. A process for producing olefins comprising contacting an organic oxygenate compound under oxygenate conversion conditions with the catalyst composition of claim 11.
